(19) United States
(12) Patent Application Publication
(10) Pub. No.: US 2024/0285338 A1
(43) Pub. Date: Aug. 29, 2024

(54) APPARATUS AND METHOD FOR DELIVERING PULSED ELECTRIC FIELD THERAPY

(71) Applicant: Pulse Biosciences, Inc., Hayward, CA (US)

(72) Inventors: Roman TUROVSKIY, San Francisco, CA (US); David J. DANITZ, San Jose, CA (US); Richard J. CONNOLLY, Riverview, CA (US)

(21) Appl. No.: 18/569,206

(22) PCT Filed: Jun. 10, 2022
(86) PCT No.: PCT/US2022/032930

§ 371 Date: Dec. 11, 2023

Related U.S. Application Data
(60) Provisional application No. 63/211,389, filed on Jun. 16, 2021.

(51) Int. Cl. A61B 18/14; A61B 17/00; A61B 18/00

(52) U.S. Cl. CPC A61B 18/1492; A61B 2017/00154; A61B 2018/00261; A61B 2018/00577; A61B 2018/1467

(57) ABSTRACT

Methods and apparatuses are disclosed for providing pulsed electrical treatment (including high voltage, sub-microsecond pulsed electric energy) to a body lumen. The methods and apparatuses of the present disclosure enhance ablation of the treatment area and/or reduce or eliminate arcing between the electrodes with a use of the filling materials.

### CROSS REFERENCE TO RELATED APPLICATIONS

This patent application claims priority to U.S. provisional patent application No. 63/211,389, titled “APPARATUS AND METHOD FOR DELIVERING PULSED ELECTRIC FIELD THERAPY,” filed on Jun. 16, 2021, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are apparatuses (e.g., devices, systems, etc.) and methods that may be used to treat a patient. Specifically, the apparatuses and methods described herein may be used to deliver short, high-field strength electrical field pulses to tissues forming a body region (e.g., a body lumen) and/or adjacent to a body lumen.

### BACKGROUND

Electric pulses, including high-field strength electric pulses have been described for electromanipulation of biological cells. For example, electric pulses may be used in treatment of human cells and tissue including tumor cells, such as basal cell carcinoma, squamous cell carcinoma, and melanoma. The voltage induced across a cell membrane may depend on the pulse length and pulse amplitude. Pulses longer than about 1 microsecond may charge the outer cell membrane and may lead to permanent opening of pores. Permanent openings may result in instant or near instant cell death. Pulses shorter than about 1 microsecond may affect the cell interior without adversely or permanently affecting the outer cell membrane and result in a delayed cell death with intact cell membranes. Such shorter pulses with a field strength varying in the range, for example, of 10 kV/cm to 100 kV/cm may trigger apoptosis (i.e. programmed cell death) in some or all of the cells exposed to the described field strength and pulse duration. These higher electric field strengths and shorter electric pulses may be useful in manipulating intracellular structures, such as nuclei, endoplasmic reticulum and mitochondria. For example, such sub-microsecond (e.g., nanosecond, picosecond) high voltage pulse generators have been proposed for biological and medical applications.

Electrical arcing happens when an electric current flows through the air between two conductors. Arcing may be particularly problematic when treating patient tissues, as the resulting arc may damage the tissue and potentially harm the patient. The risk of arcing is particularly high when applying high field strength (e.g., microsecond and sub-microsecond) pulses within regions exposed to air, such as non-vasculature body lumen, including but not limited to the lungs (bronchus, etc.) and the gastrointestinal tract (mouth, pharynx, esophagus, stomach, small intestine, large intestine, anus, etc.). However, sometimes arcing may be a risk not only when the electric current flows through the air but even in the vasculature with blood present. It would be beneficial to provide methods and apparatuses that may reduce the risk of arcing and also assist in providing uniform and effective electric field therapies from within an anatomical structure, such as a body lumen.

### SUMMARY OF THE DISCLOSURE

Described herein are apparatuses and methods for treating a biological tissue or a patient (e.g., human or animal subject) using pulsed electrical fields, including (but not limited to) sub-microsecond, such as nanosecond, pulsed electrical fields. These methods and apparatuses may be used in particular with pulsed (e.g. nanosecond pulsed) electric fields having high field strength, however, they may be used with other types of energy, for example, RF or micro-second pulsed elected field. The methods and apparatuses described herein may be configured to selectively treat a portion of a wall of a body lumen, and/or tissue adjacent to the wall of the lumen, in some examples the innermost layer(s) of the wall of a body lumen (e.g., depending on the lumen, the villus, mucosa, submucosa, surrounding muscle, etc.), with sub-microsecond (e.g., nanosecond) pulsed electrical fields in a localized manner that limits or prevents damage to deeper, non-targeted regions.

In general, the methods and apparatuses described herein may control the conductive pathway between the electrodes applying the pulsed electrical fields, including by delivering a filling material between the two (or more) electrodes applying the pulsed electrical field. The filling material may prevent or disrupt arcing between the electrodes when applying pulsed electrical fields. A gas, such as air between the electrodes may otherwise result in arcing and/or uncontrolled delivery of energy to the tissue of the lumen or surrounding tissues. Surprisingly, the methods and apparatuses described herein have also been found to enhance ablation between two (or more) electrodes, independently of any reduction in arcing. In some examples of the methods and apparatuses described herein, ablation may be enhanced by applying a conductive liquid between the electrodes within the body (e.g., within a body cavity or body lumen). The application of a conductive liquid between the electrodes may help extend ablation into the area between electrodes. In the absence of a conductive liquid ablation may otherwise be localized primarily in the region where electrodes are touching the tissue leaving the area between electrode un-ablated or less ablated.

As used herein the body lumen or lumen may refer to any anatomical structure, for example, body cavity or organ having one or more walls. The body lumen may include, but is not limited to vessels (e.g., blood vessels, arteries, etc.), ducts (e.g., urinary ducts, etc.), tracts (e.g., gastrointestinal tract, female genital tract, etc.), pathways (e.g., bronchi of lungs, etc.), tubes (renal tubules, fallopian tubes, etc.) and hollow organs (e.g., vagina, etc.).

Some methods and apparatuses described herein may be particularly well suited for use in treating body lumen that does not typically carry a conductive liquid such as the bronchial and esophageal lumina. The methods and apparatuses described herein are not limited to regions without a conductive liquid (such as blood), however. The methods and apparatuses described herein may also be used with lumen that include a conductive liquid, such as blood (e.g., arteries, blood vessels, etc.). Methods and apparatuses that apply a conductive liquid between the electrodes may apply or deliver a conductive liquid having known (controlled) electrical properties, such as conductivity, which may be specifically adapted for use with these systems. The use of an applied conductive liquid may also provide a known conductivity between the electrodes. For example, the conductivity of blood may vary. Thus, these methods and apparatuses may control the conductive pathway between the electrodes applying the pulsed electrical fields. In some examples it may also be beneficial to flush (e.g., wash, dilute, etc.) natural conductive liquid, such as blood, from the body region being treated and replace it with a filling material, such as conductive liquid having known and controlled properties (e.g., saline).

To treat these lumina, a filling material may be temporarily introduced into the lumina between and/or around the electrodes, and then the nanosecond pulsed energy treatment delivered. Following the treatment, the filling material may be removed from the lumina; in some examples the filling material may be continuously applied and removed (streamed) during the application of the high-field pulsed electrical energy within the lumen. In some cases the filling material may be a liquid (e.g., a conductive liquid) and/or a solid (e.g., a polymeric sheet, balloon or balloons, etc., expanded or inflated) applied within the lumen between the electrodes. The filling material may completely fill a portion transverse to the lumen; in some examples the filling material may completely fill the region between the electrodes.

As described here, in some examples one or more occluders (e.g., sheets, balloons, etc.) may extend across the lumen to prevent leakage or loss of the filling material from the region between the electrodes. Although in some examples it may be beneficial to block off (e.g., using a pair of occluders) the region around the electrodes to prevent migration of the filling material from between the electrodes, including potentially sealing or partially sealing off the region. In some examples only a single occluder may be used, e.g. such occlude may be positioned between the electrodes. In some examples occluders may not be used, as the filling material may remain in place long enough (or may be applied sufficiently rapidly) so that at least a portion of the region between the electrodes, and particularly a region spanning the transverse extent of the lumen (e.g., the region within the lumen or body cavity between the electrodes), is filled with the filling material while applying the electric fields. The filling material may be absorbed or cleared by the body normally, or it may be removed by the action of the apparatus or method.

In general, the electrodes may be positioned against the wall(s) of the body lumen/anatomical structure being treated. For example, the electrodes may be biased (pushed) against the wall(s) of the lumen by engaging and/or deploying one or more expandable member. The expandable member may be a mechanically expandable member such as a balloon, mesh, radially-expanding arms or struts, swellable member, etc. Each electrode, or each set of electrodes (e.g., anode and cathode) may be biased against the wall(s) of the body lumen. In some cases the expandable member(s) biasing the electrodes against the wall(s) of the body lumen may also be configured as an occluder to retain the filling material. For example, the expandable member(s) may include a surface that is substantially impermeable to the filling material, such as a sheet or layer arranged to expand with the expandable member and occlude the body lumen.

As mentioned, the methods and apparatuses described herein may be applicable to anatomical structures or lumens that typically carry a conductive liquid (such as blood), as well as lumens that may not typically carry a conductive liquid, or that do not typically include a liquid at all.

Described herein are methods and apparatuses for providing electric field treatment (e.g., pulsed sub-microsecond electric field treatment or therapy). These methods and apparatuses may be used with any body region, including (but not limited to) body regions forming a cavity or lumen. For example, a method for providing a pulsed electric field treatment may include: positioning an applicator (e.g., a treatment applicator) within a body lumen so that a first electrode of the applicator is in contact with a wall of the body lumen and a second electrode of the applicator is in contact with the wall of the body lumen; introducing a filling material within the body lumen between and/or around the first electrode and the second electrode; and applying a pulsed electric field between the first and the second electrodes.

The first and second electrodes may be positioned against the wall(s) of the body lumen by expanding one or more expandable members (e.g., balloons, mesh, etc.), as mentioned above. Thus, any of these methods may include deploying the first and second electrodes to contact the wall(s) of the body lumen. The electrodes may be deployed by deploying the expandable member(s). For example, the first electrode may be deployed by expanding or inflating a balloon to which the electrode is attached so that it expands within the body lumen and the first electrode on the balloon contacts the wall. The first electrode may have a single point of contact or it may have multiple points of contact around the body lumen circumference (e.g., annulus); for example, the first electrode may be part of multiple electrical contacts (a first set of electrodes) that may be electrically connected to form a single virtual electrode contacting the wall of the body lumen. The first electrode or set of electrodes may be coupled to the outside of an expandable member, such as a balloon, which is deployed by the user (e.g., physician, surgeon, technician, etc.) from a proximal end of the apparatus. In general, regions of the apparatus that are closer to the user operating the apparatus are considered proximal; regions further from the user operating the device are considered distal.

The electrodes may be deployed by causing the expandable member(s) to expand outwards and against the wall(s) of the body lumen including the target region to be treated. In examples in which the expandable members are a mesh, e.g., a basket or wire frame, the mesh may be expanded by releasing a constraint (if the mesh is biased, e.g., as with a shape memory material, to open in the unconstrained configuration) or by pulling proximally, e.g., a pull wire or rod coupled to the mesh (e.g., the distal end region of the mesh, with the proximal end fixed and not moving proximally) or by pushing distally (e.g., the proximal end region of the mesh, with the distal end fixed and not moving distally). Deployment of the expandable member drives the electrodes radially outward until they contact the wall(s) of the body lumen. The electrodes may be formed as part of the expandable member, including as part of the wire mesh (e.g., one or more of the wires).

In any of these methods and apparatuses, applying the pulsed electric field comprises applying a nanosecond pulsed electrical field.

In general, the filling materials that may be used herein may include any material that may disrupt and/or prevent arcing between the electrode applying the pulsed electric field energy. For example, the filling material may be one or more of: a saline liquid, a contrast dye, a hypertonic solution, a hypotonic solution, or a combination thereof. In some cases the filling material may be a conductive solution. Alternatively, in some examples the filling material may be a solid material or contained within a solid material. For example, in some implementations the filling material may include a balloon, such as a polymeric balloon, which is expanded in the body lumen between the electrodes; the balloon may be filled with saline or other material.

Any of the methods, or apparatuses configured to perform these methods, may monitor to confirm that the filling material has filled the space within the body lumen between the first electrode and the second electrode, or that a circumference of the body lumen at least in a portion of the length between the first and the second electrode is occluded. For example, the methods or apparatuses may detect the pressure within the body lumen between the first electrode and the second electrode; an increase in pressure to a threshold may indicate that the region has been filled, particularly in examples in which the region of the body lumen around the electrodes is occluded. In some examples the methods or apparatuses may detect an impedance, e.g. of the region in the body lumen between the electrodes; the impedance may be predicted based on the known electrical properties of the filling material (e.g., conductive solution). In some cases separate electrodes on the applicator may be used to detect the impedance; in other examples, one or both electrodes used to deliver the pulsed electrical energy may be used (including used before being driven against the body lumen wall(s)). In some examples the methods or apparatuses may optically detect that the filling material has filled the region between the electrodes. For example, filling material may include a contrast dye that may be detected within the body lumen between the first electrode and the second electrode. The filling material may be detected using an optical channel within the apparatus or externally (e.g., using fluoroscopy, etc.).

In any of these methods and apparatuses, delivering the filling material may comprise blocking (e.g., disrupting and/or preventing formation of) an air path between first electrode and the second electrode with the filling material. As mentioned, in some examples the method may include delivering a conductive liquid from out of an outlet (e.g., infusion port) on the applicator. In some examples delivering the filling material comprises expanding inflatable member within the body lumen between the first electrode and the second electrode such that the inflatable member blocks an air path between the first electrode (or set of first electrodes) and the second electrode (or set of second electrodes).

In any of these methods the region of the body lumen including the electrodes may be blocked off. For example, any of these methods may include occluding the body lumen prior to delivering the filling material.

Occluding the body lumen may comprise expanding a first member to which the first electrode is coupled and expanding a second member to which the second electrode is coupled.

As mentioned above, in some examples positioning the applicator comprises expanding a first member to which the first electrode is coupled and expanding a second member to which the second electrode is coupled.

These methods may be used to treat any appropriate body lumen. For example, positioning the applicator may include positioning the applicator within one of: a blood vessel, a bronchus, a gastrointestinal tract, a urethra, a fallopian tube, a trachea, an esophagus, or a sinus. The body lumen may be within or coupled to the respiratory anatomy, including one or more of: the nose, mouth, pharynx, larynx, trachea, large airways (bronchi) or small airways. The body lumen may be within or coupled to the gastrointestinal anatomy (alimentary canal), including one or more of: mouth, esophagus, stomach, large intestine (cecum, appendix, ascending colon, transverse colon, descending colon, sigmoid colon), small intestine (duodenum, jejunum, ileum), colon, rectum, anus, etc. The body lumen may be within or coupled to the female reproductive tract including one or more of: vagina, cervix, uterus, uterine tube/fallopian tube, etc. The body lumen may be within or coupled to the urinary tract, including: bladder, ureter, urethra, etc. The body lumen may be within or coupled to the circulatory system, including one or more of: arteries, veins, heart (e.g., superior vena cava, aorta, atrium, ventricle, pulmonary vein, etc.). The body lumen may be within or coupled to the ear canal.

Any of these methods may include removing the filling material from the body lumen after (or during) the application of the pulsed electric fields. In some examples filling material may be continuously delivered as the pulsed electrical field is applied. In some examples filling material may be continuously delivered and removed as the pulsed electrical field is applied.

According to some aspects of the present disclosure, a method of enhancing ablation or controlling a conductive pathway between electrodes is provided. Such method may include: positioning an applicator within a body lumen at a treatment region; expanding one or more expandable members at a distal end region of the applicator so that a first electrode is in electrical contact with a wall of the body lumen and a second electrode is in contact (e.g., in electrical contact) with the wall of the body lumen; applying a sub-microsecond pulsed electric field between the first and second electrodes to treat the tissue forming or adjacent to the body lumen; and delivering a conductive liquid within the body lumen between the first electrode and the second electrode thereby enhancing ablation and/or controlling a conductive pathway between the first and the second electrodes. The step of delivering a conductive liquid within the body lumen may be performed in some implementations before or during application of the sub-microsecond pulsed electric field.

Also described herein are apparatuses that may be used in any of the methods described herein. These apparatuses may include devices (e.g., applicators, pulse generators, etc.) and systems configured for delivering electric treatment, such as pulsed electrical fields. For example, an apparatus may include: an elongate applicator body; a first expandable member at a distal end region of the elongate body; a first electrode on or forming at least a part of the first expandable member; a second expandable member at the distal end region of the elongate body and laterally offset from the first expandable member; a second electrode on or forming at least a part of the second expandable member; and an outlet (e.g., an infusion port) configured to deliver a filling material between the first electrode and the second electrode.

The expandable member may be a balloon, mesh, extendable arm(s) or struts, etc., as described herein. As mentioned, in examples having more than one expandable member, the expandable members may be the same or different; for example, one expandable member may be an expandable mesh (knit, woven, braided, etc.) while a second expandable member may be a balloon.

Any of these apparatuses may include an occluder. The occluder generally occludes the body lumen to prevent the filling material from moving out of the region between the electrodes. One or more occluder may be used. For example, the apparatus may include a pair of occluders on either side of the electrodes. The occluder typically includes an expandable sealing surface (e.g., a first expandable sealing surface) configured to occlude the body lumen when deployed in the body lumen. For example, the sealing surface may be at a distal end region of the elongate body, e.g., distal and/or proximal to the first electrode and second electrode. As mentioned, in some examples the apparatuses include a single occluder. A single occluder may be helpful, for example, where gravity or the anatomy may prevent the filling material from leaving the region of the body lumen between the electrodes.

In some examples, the expandable member(s) that position the first and/or second electrodes against the wall(s) of the body lumen may be configured to occlude. For example, one or both of the first expandable member and the second expandable member may be a balloon having a surface that occludes the vessel. One or both of the first expandable member and the second expandable member may comprise a mesh onto which a sealing surface is attached to occlude the body lumen. Thus, the first and/or second expandable sealing surfaces may be formed on the first and/or second expandable members. Thus, the second expandable sealing surface may be formed on the second expandable member. As mentioned, the occluder may be a balloon; thus, the first expandable sealing surface may include balloon. For example, one or both of the first expandable member and the second expandable member may include a balloon.

In some examples the apparatus may include a second expandable sealing surface at a distal end region of the elongate body that is laterally offset from the first expandable sealing surface so that the first and second electrodes are between the first and second sealing surfaces and the first and second sealing surfaces form an enclosed space within the body lumen when deployed in the body lumen.

Also described herein are apparatuses and methods in which the region of the body lumen between the first and second electrodes is occluded (for example, by an expandable member, such as a balloon or other expandable/deployable sealing membrane). Thus, in any of these variations an expandable member may be positioned between the first and second electrodes. The expandable member may include a balloon configured to be inflated. The inflated balloon may therefore occlude and prevent arcing between the electrodes in the body lumen.

The elongate applicator body may be a catheter.

Any of the apparatuses described herein may include an inlet for removing the filling material between the first electrode and the second electrode. Alternatively the outlet may be configured to also serve as an inlet. The outlet and/or inlet may be coupled to a channel through the apparatus extending proximally. In various implementations the outlet may comprise one or a plurality of outlets and at least one of the outlets may be positioned on the first expandable member, or on the second expandable member, or along a length of the elongated body between the first expandable member and a second expandable member, Any of these apparatuses may include a reservoir of filling material (e.g., a conductive liquid, etc.) in liquid communication with the one or more outlets. As mentioned, in some examples the filling material may be one or more of: a saline, a contrast dye, a hypertonic solution, a hypotonic solution, a solid material, a generally conductive liquid, or a combination thereof.

Any of these apparatuses may include a pulse generator configured to deliver electrical energy, such as pulsed electric fields, to the applicator. The pulse generator may include a controller (or the controller may be a separate component coupled to the pulse generator). The controller may coordinate the deploying of the filling material, optionally confirming that the filling material is sufficiently deployed between the electrodes within the body lumen (e.g., blocking any air gap between the two), and/or applying the pulsed electric fields from the electrodes. For example, the controller may be configured to deploy the filling material out of the outlet of the applicator, and further configured to deliver the pulsed electrical energy between the first and second electrodes. In any of these apparatuses the pulse generator may be configured to apply a nanosecond pulsed electrical energy. As mentioned, the controller may be further configured to confirm that the filling material has filled a space within a body lumen between the first electrode and the second electrode or that a target amount of the filling material was introduced into the treatment area. For example, the controller may be configured to confirm that the filling material has filled the space, for example, by detecting one or more of: a pressure, an impedance, or a contrast dye. The controller may be configured to coordinate the application of pulsed electrical energy so that the pulsed electrical energy is applied after the filling material has been deployed from out of the applicator.

For example, an apparatus may include: an elongate catheter; a first expandable member at a distal end region of the elongate catheter; a first electrode on the first expandable member; a second expandable member at the distal end region of the elongate catheter and laterally offset from the first expandable member; a second electrode on the second expandable member; and an outlet configured to delivering a conductive liquid filling material between the first electrode and the second electrode.

The apparatuses described herein may include or may otherwise be implemented as an applicator for delivering a pulsed electric field treatment to a patient. For convenience of description any reference to a “patient” shall be understood to include any human or animal subject, as well as any biological tissue or cells from a human or animal subject (such as samples, including biopsies from a patient). The applicator may include an elongate body (e.g., a rod or catheter), a first expandable member coupled to the catheter, a first inlet (e.g., an infusion port) disposed on the catheter and configured to infuse and in some variations remove a conductive liquid from a treatment area within a body lumen, and a first electrode on or forming a part of the first expandable member, wherein the first expandable member is configured to expand and contact the first electrode (or set of electrodes) to an inner wall of the body lumen in the treatment area and wherein the first electrode is configured to deliver electrical treatment, e.g., a nanosecond pulsed electrical treatment, while the conductive liquid is present. In some variations, the conductive liquid may include at least one of a saline liquid, a contrast dye, a hypertonic solution, a hypotonic solution, or a combination thereof. The conductive liquid may enhance tissue ablation associated with the nanosecond pulsed electrical field treatment.

In some embodiments, the first expandable member may be configured to form a leak-resistant seal (e.g., to form or include the occluder) with the inner wall of the lumen. Alternatively, a separate occluder may be positioned proximally or distally of the electrodes on the expandable member. In some other variations, the first expandable member may be configured to restrict a flow of the conductive liquid in the lumen.

In some examples the first expandable member may be configured to be expanded by a fluid (which in some examples may be a conductive liquid). Furthermore, the first expandable member may include a second outlet configured to infuse and remove the fluid from the treatment area.

In some variations, the first electrode or the first set of electrodes may be disposed around the first expandable member. In some other variations, the applicator may include a second expandable member coupled to the elongate member (e.g., catheter) and configured to expand. The second expandable member may be configured as a part of a second occluder. For example, the second expandable member may include a membrane that forms a leak-resistant seal with the inner wall of the body lumen. Furthermore, the first expandable member and the second expandable member may be configured to retain the conductive liquid within the treatment area. The treatment area may be between the first expandable member and the second expandable member. In some variations, contact between the body lumen and the first expandable member and the second expandable member may be verified by one or more of: a pressure measurement of the conductive liquid, visually confirming, and/or by sensing an electrical property (e.g., impedance). The method may be suspended or modified if contact between the expandable member(s) and the body lumen is not verified. Alternatively or additionally the method or apparatus (e.g., system) may verify that the treatment region is sealed off, or at least partially isolated so that the region may be filled with a conductive liquid (or confirmed to be filled with a conductive liquid) as described herein.

In some examples, the first outlet may be disposed between the first expandable member and the second expandable member. In some examples, the applicator may include a second electrode configured to expand and contact the inner wall of a lumen within the treatment area. A nanosecond pulsed electrical field treatment may be applied between the first and second electrodes while the conductive liquid is present. Contact with the inner wall of the lumen may be verified as mentioned above, including (but not limited to) measuring an impedance measurement between the first electrode and the second electrode. The second electrode may be disposed around the second expandable member. The second expandable member may be configured to contact the second electrode (or set of electrodes) to the inner wall of the lumen in response to an expansion of the second expandable member. In some variations, the applicator may include an optical assembly configured to verify contact of the first expandable member and the lumen.

The methods for providing a pulsed electric field treatment via an applicator described herein may include positioning the applicator in a treatment area within a lumen, expanding one or more expandable members to contact the lumen in the treatment area, infusing the treatment area with a conductive liquid, and providing, by one or more electrodes, a nanosecond pulsed electrical field treatment to the lumen while the conductive liquid is present.

In some variations, the method may also include draining the conductive liquid from the treatment area and collapsing the one or more expandable members so that the electrodes are withdrawn from the walls. In some variations, the conductive liquid may include at least one of a saline liquid, a contrast dye, a hypertonic solution, a hypotonic solution, a solid material (e.g., balloon), or a combination thereof. The conductive liquid may be configured to enhance tissue ablation associated with the nanosecond pulsed electrical field treatment and/or reduce or eliminate arcing.

In some variations, the one or more expandable members may restrict a flow of the conductive liquid in the body lumen. In some other variations, the one or more electrodes may be disposed around the one or more expandable members.

In some variations, the one or more electrodes may include a first electrode and a second electrode, and the nanosecond pulsed electrical field treatment may include bipolar electric fields between the first and second electrodes.

The one or more expandable members may be actuated by the user at a proximal handle. In some examples, the one or more expandable members may expand in response to a pressure provided by the conductive liquid. The conductive liquid may be provided through a catheter.

For example, described herein are applicators for delivering a pulsed electric field. The applicator may include an elongate body (e.g., a catheter), a first expandable member coupled to the catheter and configured to expand and contact an inner wall of a body lumen, a second expandable member coupled to the catheter and configured to expand and contact the inner wall of the body lumen, a first electrode or set of electrodes on the first expandable member or forming at least a part the first expandable member, a second electrode or set of electrodes on the second expandable member or forming at least a part of the second expandable member, and a third expandable member coupled to the elongate body and positioned between the first electrode or set of electrodes and the second electrode or set of electrodes, wherein the third expandable member is configured to eliminate or reduce electrical arcing between the first and the second electrodes. The third expandable member may be configured as an occluder to expand and to be filled with the filling material, for example, saline, such that when expanded, the third expandable member contacts the wall of the body lumen and forms a leak-resistant barrier or a seal with the wall of the body lumen, thereby preventing or at least reducing arcing between the electrodes and/or enhancing ablation. The third expandable member may comprise a balloon. The first and the second electrodes may be configured to deliver a nanosecond pulsed electrical field treatment to the lumen.

As used herein, when it is described that a first object may be in contact (including in electrical communication) with a second object, the first object may contact the second object either directly or indirectly and may communicate a force thereon. For example, the first object may directly contact the second object when at least a portion of the first object touches the second object. The first object may indirectly contact the second object when an intermediate object is positioned between the first object and the second object so that the first object may communicate a force to the second object through the intermediate object. For example, a first object (e.g., electrode) may be in electrical communication with a second object (e.g., the wall of the body cavity or lumen) either directly or indirectly, so that the electrical energy applied to the first object may be received by the second object without substantially modifying the electrical energy (or in some cases without modifying the electrical energy at all).

In some variations, the expandable member may be configured to expand and contact the inner wall of the lumen. In some other variations, the first and the second electrodes may be configured to circumferentially contact the inner wall of the lumen. Furthermore, the catheter may include an outlet configured to expand and contract the expandable member. Contact between the expandable member and the lumen may be verified by a pressure measurement.

In some variations, the applicator may include an optical assembly configured to verify contact of the expandable member(s) and the lumen. Further, according to some examples, an apparatus for delivering pulsed electrical fields within a body lumen may comprise: an elongate body; a first expandable member at a distal end region of the elongate body; one or more electrodes on the first expandable member; an occluder at the distal end region of the elongate body and laterally offset from the first expandable member; and an outlet configured to deliver a filling material between one or more electrodes of the first expandable member and the occluder.

Another aspect of subject matter described herein may be implemented as a method for providing a pulsed electric field treatment via an applicator. Such method may reduce or eliminate arcing between one or more first electrode and one or more second electrodes. The method may include positioning an applicator comprising a first expandable member and a second expandable member in a treatment area, expanding the first and the second expandable members so that electrodes of the first and the second expandable members contact an inner wall of a lumen in the treatment area, wherein the first and second expandable members and electrodes are coupled to an elongate body (e.g., a catheter), expanding a third expandable member disposed between the first and the second electrodes with a filling material such that the third expandable member interrupts a conductive path or blocks an air path between the electrodes, and applying a nanosecond pulsed electrical field treatment through the first and the second electrodes.

In some variations, the third expandable member may be configured to circumferentially contact the lumen between the electrodes (or sets of electrodes). In some other variations, the electrodes (or sets of electrodes) may be configured to circumferentially contact the inner wall of the lumen. In still other variations, expanding the third expandable member may be in response to an infusion of a liquid or a gas into an interior of the third expandable member. The method may include measuring a pressure of the liquid or the gas and verifying contact between the third expandable member and the lumen based at least in part on the measured pressure.

Any appropriate conductive liquid may be used, including but not limited to saline (such as, but not limited to, a 0.9% saline solution). The electrical conductivity (x) of the conductive liquid may be, for example, between about 2 mS/cm and about 50 mS/cm at 25 degrees C. (e.g., between about 5 mS/cm and about 45 mS/cm, between about 5 mS/cm and about 40 mS/cm, between about 10 mS/cm and about 30 mS/cm, between about 5 mS/cm and about 20 mS/cm, about 10 mS/cm, about 12 mS/cm, about 15 mS/cm, about 16 mS/cm, about 18 mS/cm, etc.)

All of the methods and apparatuses described herein, in any combination, including a combination of various features disclosed in reference to various examples, are herein contemplated and can be used to achieve the benefits as described herein.

### BRIEF DESCRIPTION OF THE DRAWINGS

The novel features of the present disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 illustrates one example of a system for delivering high voltage, sub-microsecond (e.g., nanosecond) pulses of electrical energy.

FIG. 2 illustrates an example applicator for delivering electrical field treatment according to some embodiments.

FIG. 3 shows another example of an applicator.

FIG. 4 shows another example of an applicator.

FIG. 5 shows another example of an applicator.

FIG. 6A illustrates one example of a model lumen tissue in which a conductive liquid was applied between a pair of electrodes using an apparatus similar to that shown in FIG. 3 and sub-microsecond pulsed energy was applied. FIG. 6B shows an example of a model lumen tissue similar to that shown in FIG. 6A, in which the same treatment energy was applied but without filling the treatment region with conductive liquid.

FIG. 7 is a flowchart depicting an example of one method for applying a pulsed electrical treatment to a patient.

FIG. 8 is a flowchart depicting another example of a method for applying a pulsed electrical treatment to a patient.

### DETAILED DESCRIPTION

Described herein are systems and methods for applying pulsed electrical field using electrodes adapted to be inserted into a body lumen such as, for example, esophageal, bronchial and other vessels, including vessels that may not typically convey a conductive liquid such as blood, or the like. These apparatuses and methods may prevent or reduce arcing, while providing effective treatment to the wall(s) of a body lumen and/or regions adjacent to the walls.

The electrical treatment may be, for example, microsecond pulsed treatment, or sub-microsecond pulsed treatment, including nanosecond pulses. For example, nanosecond pulsed electric fields treatment may refer to the application of relatively high voltages (in some cases 5 kV or greater) for a relatively short amount of time (in some cases less than 1 μs, such as between about 999 nanoseconds and 1 ns, etc.). These high voltages and short duration times create a pulsed electric field in the region that the voltages are applied. In some cases, electric treatment, e.g., nanosecond pulsing, may induce apoptosis or regulated cell death within cellular structures in a controlled and effective manner.

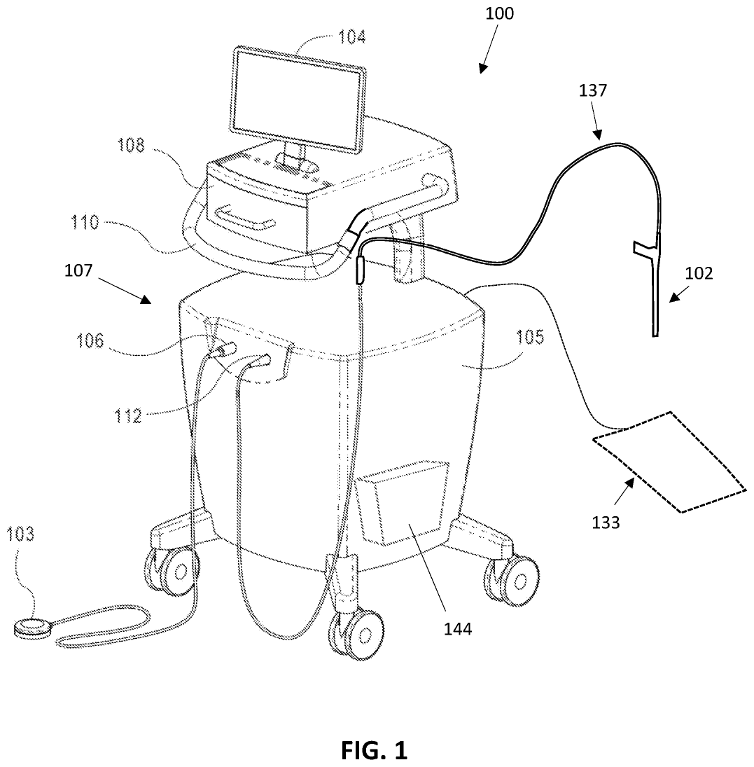

FIG. 1 illustrates one example of a system 100 (also referred to herein as a high voltage system or a sub-microsecond generation system) for delivering high voltage, fast pulses of electrical energy that may include an elongate applicator tool 102, a pulse generator 107, footswitch 103, and user interface 104. Footswitch 103 is connected to housing 105 (which may enclose the electronic components) through a cable and connector 106. The elongate applicator tool 102 may include electrodes and is connected to housing 105 and the electronic components therein through a cable 137 and high voltage connector 112. The high voltage system 100 may also include a handle 110 and storage drawer 108. The system 100 may also include a holder (e.g., holster, carrier, etc.) (not shown) which may be configured to hold the elongate applicator tool 102. In some examples the system may be configured for monopolar treatment and may optionally include a dispersive electrode 133 (e.g., a return electrode pad).

In some cases, the elongate applicator tool 102 includes one or more imaging sensors, such as one or more cameras and/or fiber optics at or near the distal end of the elongate applicator tool 102. The camera(s) (not shown for simplicity) may be forward-facing and/or side facing. The system 100 may be configured to display images (in real time, and/or recorded) taken by the elongate applicator tool 102, in order to identify the target treatment region(s).

A human operator may select a number of pulses, amplitude, pulse duration, and frequency information, for example by inputting such parameters into a numeric keypad or a touch screen of interface 104. In some examples, the pulse width can be varied. A controller 144 (e.g., microcontroller) may send signals to pulse control elements within the system 100. In FIG. 1, the controller (which may include one or more processors and other control circuitry, including memory) is shown within the housing 105, but it may be positioned anywhere in the system. The controller may be coupled to the pulse generator and/or power supply and may receive input from any of the input components. One or more processors (not shown) may be a separate processing unit or may be incorporated with the controller. The controller may comprise a plurality of controllers and the processor may comprise a plurality of processors. In some examples, fiber optic cables are used which allow control signaling while also electrically isolating the contents of the metal cabinet (e.g., the housing 105) with a sub-microsecond pulse generation system 100, e.g., the high voltage circuit, from the outside. In order to further electrically isolate the system, system 100 may be battery powered instead of being powered from a wall outlet.

The elongate applicator tool 102 may be hand-held (e.g., by a user) or it can be affixed to a movable arm of a robotic system, and its operation may be at least partially automated or fully automated, including computer controlled operation.

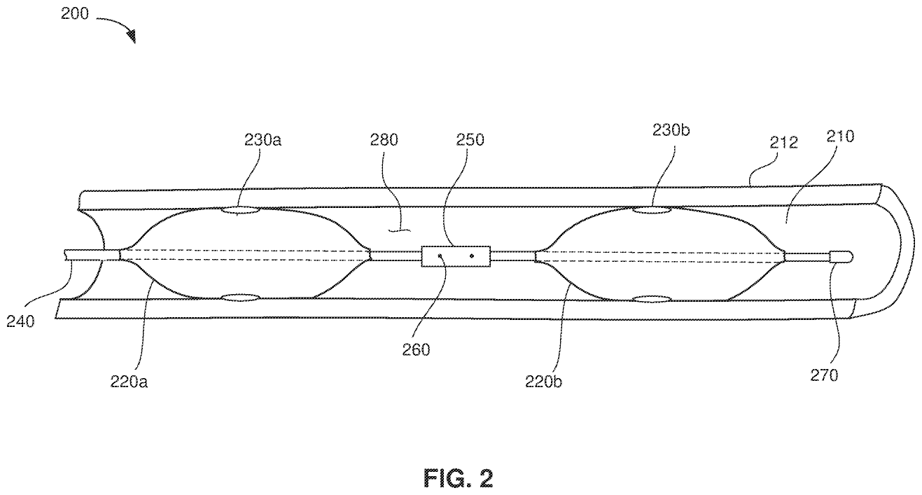

FIG. 2 illustrates an example of an applicator 200 (which may be also referred to as a treatment applicator) for delivering a nanosecond pulsed electrical field treatment within a body lumen 210 (having a wall 212, in accordance with some embodiments. The lumen 210 may be any feasible lumen including, but not limited to, esophageal, bronchial, pulmonary, and arterial lumina. In this example, the applicator 200 may include a proximal (e.g., first) expandable member 220a, a distal (e.g., second) expandable member 220b, a proximal electrode 230a, a distal electrode 230b, an elongate body (e.g., catheter) 240, an optional sleeve 250 or similar structure, one or more outlets/inlets (e.g., infusion ports) 260, and an atraumatic tip 270. Although the applicator 200 is shown with two expandable members 220a and 220b, two electrodes 230a and 230b, and one sleeve 250, the applicator 200 may include any feasible number of expandable members, electrodes, and sleeves. In particular, the apparatus may include a first set of electrodes including the first electrode and a second set of electrodes, including the second electrode, which may be arranged circumferentially around the respective expandable members. The applicator 200 may be coupled (for example, by the elongate body 240) to the applicator tool 102 (e.g., including a handle and/or attachment) and thereby coupled to the system 100 of FIG. 1. “Proximal” may refer to elements nearer to the user's body when operating the applicator tool 102 (e.g., toward the left of FIG. 2) and “distal” may refer to elements further from the user (e.g., toward the right of FIG. 2).

The proximal and distal expandable members 220a and 220b (in some examples, balloons) may be coupled to the elongate body and formed from any feasible, conformable, and expandable material. In at least one example, the proximal and distal expandable members 220a and 220b may be inflated, enlarged, or expanded by any feasible liquid (e.g., liquid or gas). The liquids or gas used for inflation may be passed through the elongate body (e.g., catheter) 240. In some variations, one or more outlets (and/or inlets) may be disposed on the catheter 240 to inflate the proximal and distal expandable members 220a and 220b. Thus, the proximal and distal expandable members 220a and 220b may be balloons inflated by liquid provided through the catheter 240. In another example, the proximal and distal expandable members 220a and 220b may be expanded by pressure from any feasible liquid. In some embodiments, contact between the proximal and distal expandable members 220a and 220b and the lumen 210 may be verified by measuring or monitoring the pressure of the inflation liquid. A measured pressure greater than a threshold may indicate that a positive seal exists between the proximal and distal expandable members 220a and 220b and the lumen 210.

The proximal and distal electrodes 230a and 230b may be formed from any feasible conformable material including, but not limited to, woven and/or braided wire, conductive traces, conductive ink, discrete wires, and the like. The proximal and distal electrodes 230a and 230b may be attached, bonded, disposed around, surround, or otherwise coupled to the proximal and distal expandable members 220a and 220b, respectively. In other embodiments, the proximal and distal electrodes 230a and 230b may be disposed adjacent to, but not attached to, the distal and proximal expandable members 220a and 220b. The proximal and distal electrodes 230a and 230b may be coupled through the elongate body 240 to the system 100 (coupling wires and connections not shown for clarity).

In some examples, the electrodes may themselves be expandable electrodes. For example, the electrodes may be formed of a mesh or other material that is arranged on the expandable member (or that forms all or part of the expandable member) and may be expanded or collapsed as described herein in general for the expandable members.

The (optional) sleeve 250 may be disposed on the elongate body, such as cannula or catheter 240. Furthermore, the one or more outlets and/or inlets 260 may be disposed on the sleeve 250 or simply on the elongate body 240 itself (e.g., cannula or catheter). The sleeve 250 may provide a controllable and stable platform to support the outlets and/or inlets 260. Although only two outlets and/or inlets 260 (e.g., infusion ports) are shown, any feasible number of outlets/inlets 260 may be included. In some embodiments, the outlets/inlets 260 may be disposed directly into the catheter 240 (removing or lessening the need for the sleeve 250).

The atraumatic tip 270 may be disposed on or near the distal end of the applicator 200 to limit and/or prevent abrasion, irritation and/or injury to the patient as the applicator 200 is positioned within the lumen 210 to provide treatment.

In some examples, the applicator 200 may be guided to the identified treatment region by the elongate body 240 and a proximal handle (such as the elongate applicator tool 102 in FIG. 1). Furthermore, the applicator 200 may also be guided to a treatment area in the lumen 210 by guide wires (not shown) and/or the use of fluoroscopy equipment. The applicator 200 may include a central lumen (also not shown) which may allow it to be delivered over a guide wire. Alternatively, a rapid exchange lumen may be present on a side of the applicator distal end.

After the applicator 200 is positioned within the lumen 210, the position of the applicator 200 may be verified, and the proximal and distal expandable members 220a and 220b may be expanded to conform to an inner wall of the lumen 210. The proximal and distal expandable members 220a and 220b may ensure that the proximal and distal electrodes 230a and 230b are brought into positive contact with the inner wall of the lumen 210. In some variations, the proximal and distal electrodes 230a and 230b (or sets of electrodes) may circumferentially or approximately circumferentially contact the inner wall. Additionally, in some examples the proximal and distal expandable members 220a and 220b may provide a leak-resistant barrier between the respective expandable member and the inner wall of the lumen 210. Thus, the expandable members may be configured as occluders. Alternatively, in some examples, the proximal and/or distal expandable members may be formed of a non-occluding material, such as a wire mesh (e.g., a knitted, woven, or braided filament mesh), as will be shown in some of the other examples, below. In FIG. 2, in variations in which the expandable electrodes 230a, 230b are sealed against the wall of the lumen by the expandable member (e.g., balloons 220a, 220b), it may not be necessary to fill the space between the electrodes with a conductive liquid. However, in some examples it may still be beneficial, for example, if a portion of the electrodes is not completely sealed or is exposed to the lumen (and particularly the portion of the lumen between the occluders), which may result in arcing and/or less uniform distribution of energy to the tissue, as described herein.

A filling material, such as but not limited to a conductive liquid 280, may be introduced (e.g., infused) into a treatment area through the outlets and/or inlets 260. For example, a pump may be included in the system 100 that may pump the filling material 280 through the outlets and/or inlets 260. Notably, larger numbers of outlets and/or inlets 260, and/or larger diameter outlets and/or inlets may enable a faster introduction of filling material 280 to the treatment area. The filling material may be saline, a contrast dye, a mix of saline and contrast dye, a hypertonic solution, a hypotonic solution, or a generally conductive liquid. In some embodiments, the filling material 280 may also be used to inflate the proximal and distal expandable members 220a and 220b.

In some examples, a desired level of filling material between the proximal and distal expandable members 220a and 220b and the lumen 210 may be verified. For example, the presence of a target amount of filling material may be determined by measuring and/or monitoring a pressure of the filling material 280. For example, the system 100 may include a pressure transducer configured to measure pressure of the filling material 280 provided, at least in part, by a fluid pump. A measured pressure greater than a threshold may indicate that a positive seal exists between the proximal and distal expandable members 220a and 220b and the lumen 210 and that the filling material is included to a desired level. In some other variations a low voltage and/or low current pulse signal may be provided to the proximal and distal electrodes 230a and 230b or to separate sensing electrodes between these distal and proximal electrodes (or sets of electrodes). In this manner, an electrical property, such as an impedance, as influenced by the presence of the filling material within the body lumen region between the sensing and/or proximal and distal electrodes, may be determined. Contact between the proximal and distal electrodes 230a and 230b and the lumen 210, may also be confirmed electrically (e.g., by impedance sensing); in some examples this may also confirm contact and/or a seal between the proximal and distal expandable members 220a and 220b and the lumen 210.

In some embodiments, the applicator 200 may include a camera, a remote vision apparatus, or other feasible optical assembly (not shown) to provide images of the applicator 200 within the lumen 210. After the proximal and distal expandable members 220a and 220b are expanded, the camera (or other optical device) may be used to verify the filling material is filled to a desired level.

Since in some examples the proximal and distal expandable members 220a and 220b may form leak-resistant seals with the wall of the lumen 210, the filling material 280 may remain localized or restricted to the treatment area (e.g., the area between the proximal and distal expandable members 220a and 220b). In some cases, the filling material 280 may enhance tissue ablation associated with a nanosecond pulsed electrical field treatment provided by the proximal and distal electrodes 230a and 230b within the treatment area. Thus, presence of the filling material 280 may be advantageous. In some variations, the filling material 280 may prevent or reduce electrical arcing between the proximal and distal electrodes 230a and 230b. The presence of the filling material may also provide controlled electrical properties (e.g., conductivity) between the electrodes, as well as contribute to providing uniform and effective electric field.

After the region between the proximal and distal expandable members 220a and 220b has been infused with the conductive liquid 280, nanosecond pulsed electrical field treatment may begin. In general, the pulsed electric field energy is applied between the first and second electrodes. In some examples, optionally, the applicator 200 may be configured for bipolar operation, e.g., operation where pulse energy is transferred between the proximal and distal electrodes 230a and 230b with different polarities. For example, the proximal electrode 230a may be associated with a signal having first polarity (e.g., a positive signal) and the distal electrode 230b may be associated with a signal having second polarity (e.g., a negative signal). In other examples, the proximal electrode 230a may be associated with a signal having a negative signal and the distal electrode 230b may be associated with a signal having a positive signal.

After completion of the delivery of the nanosecond pulsed electrical field treatment, the filling material 280 may be removed from the treatment area. For example, the conductive liquid pump may withdraw the filling material 280 from the treatment area through the outlets and/or inlets 260. After the filling material 280 has been removed, the proximal and distal expandable members 220a and 220b may be compressed (e.g., deflated or collapsed) and the applicator 200 may be moved to another area of the lumen 210 or removed from the patient.

Distance between the proximal and distal expandable members 220a and 220b, and therefore distance between proximal and distal electrodes 230a and 230b, may vary based on any particular embodiment of the applicator 200. In some examples, a greater distance between the proximal and distal expandable members 220a and 220b may increase the treatment area while decreasing a voltage density per unit area. On the other hand, a lesser distance between the proximal and distal expandable members 220a and 220b may decrease the treatment area and increase a voltage density per unit area. In some embodiments, the distance between the proximal and distal expandable members 220a and 220b may be determined at least in part by a length associated with the sleeve 250.

In some variations, the applicator 200 may include a single electrode or a set of electrodes of the same polarity (not shown). For example, the applicator 200 may be used to provide monopolar nanosecond pulsed electrical field treatment using either a single electrode or a set of electrodes of the same polarity. Also, in some variations, the applicator 200 may include one expandable member comprising one or more electrodes (e.g., similar to the expandable member 220a) In this implementation, for example, two additional expandable occluders (such as balloons) may be positioned distally and proximally from the electrode(s) of the one expandable member. The volume between the two additional expandable occluders may be filled with the fillable material, for example, through one or more inlets/outlets. The one or more outlets/inlets may be positioned, for example: 1) on the one expandable member with electrodes, and/or 2) along the elongated body between the one expandable member with electrodes and the first of the two expandable occluders, and/or 3) along the elongated body between the one expandable member with electrodes and the second of the two expandable occluders.

In one example, the applicator 200 may be used within the bronchial system. The applicator 200 may be placed through an endotracheal tube positioned proximal to a bronchial carina. The applicator 200 may be positioned at the treatment area with a bronchoscope or other feasible means. The proximal and distal expandable members 220a and 220b may be expanded and the conductive liquid 280 infused through the outlets and/or inlets 260. Nanosecond pulsed electrical field treatment may be delivered through the proximal and distal electrodes 230a and 230b. Following treatment, the conductive liquid 280 may be pumped out of the treatment area and the proximal and distal expandable members 220a and 220b may be collapsed or retracted. The applicator 200 may be moved to another treatment area or withdrawn from the patient.

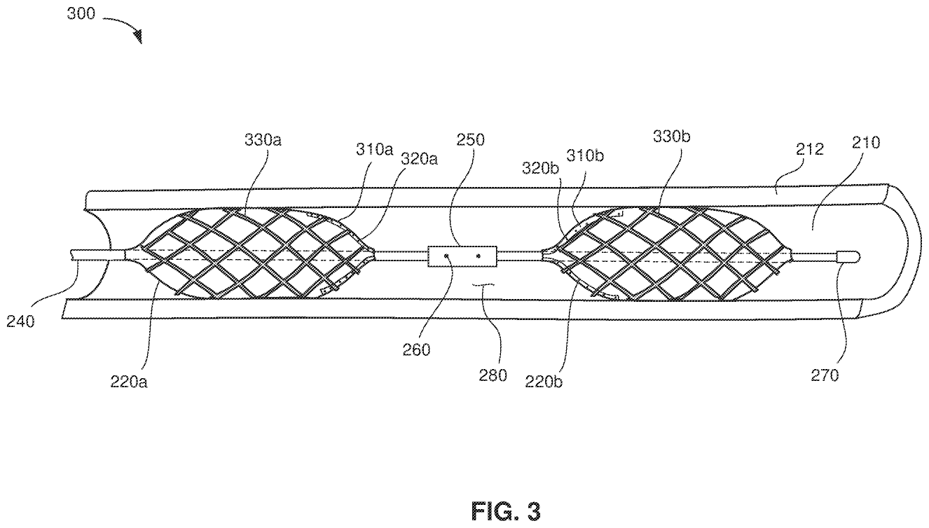

FIG. 3 shows another example of an applicator (e.g., treatment applicator) 300, in accordance with some embodiments. Similar to the applicator 200 of FIG. 2, the applicator 300 may include a proximal expandable member 220a, a distal expandable member 220b, a proximal electrode 330a, a distal electrode 330b, a catheter 240, and an atraumatic tip 270. In addition, the applicator 300 may include a plurality of inlets and/or outlets 320a, 320b on the expandable members; in FIG. 3 this is shown as a first perforated tube 310a disposed on a distal portion of the proximal expandable member 220a and a second perforated tube 310b disposed on a proximal portion of the distal expandable member 220b. The first perforated tube 310a may include one or more outlets and/or inlets 320a and the second perforated tube 310b may include one or more outlets and/or inlets 320b. In addition, in FIG. 3 the apparatus also includes an optional sleeve 250 and one or more (optional) outlets and/or inlets 260 on the sleeve. Any of these outlets and/or inlets may be configured as outlets for releasing liquid (e.g., conductive liquid) and/or inlets for removing liquid (e.g., blood, conductive liquid, etc.).

Similar to as described with respect to the applicator 200, the applicator 300 may be positioned within the lumen 210 having a wall 212. The proximal and distal expandable members 220a and 220b may be inflated and/or expanded to form a leak-resistant seal to the lumen 210. The first and second perforated tubes 310a and 310b may be coupled to the catheter 240 and may infuse and/or remove the filling material (e.g., conductive liquid) 280 to and from the treatment area along with, or in alterative to, the outlets and/or inlets 260. The outlets and/or inlets 320a and 320b may enable the filling material 280 to be introduced and removed more quickly from the treatment area compared to being limited to the outlets and/or inlets 260. After infusion of the conductive liquid 280, a pulsed electrical field treatment may be provided through the proximal and distal electrodes 330a and 330b. Following the pulsed (e.g., sub-microsecond) electrical field treatment, the conductive liquid 280 may be removed through the outlets and/or inlets 260 and/or the outlets and/or inlets 320a and 320b. Operation of the applicator 300 may otherwise be similar to the operation of the applicator 200 as described with respect to FIG. 2.

In the example shown in FIG. 3 the first electrode 330a and the second electrode 330b are shown as wires formed as part of a mesh on each expandable member 220a, 220b. These electrodes may be expandable with (or independently of) the expandable members and may make contact with the lumen of the vessel. All or a portion of the mesh forming the first or second electrodes may be insulated. For example, the region of mesh forming the first electrode that is proximal and/or distal to the midline region of the first expandable member (e.g., balloon) may be insulated so that the active region of the electrode is limited to this midline region. The second electrode may be configured similarly.

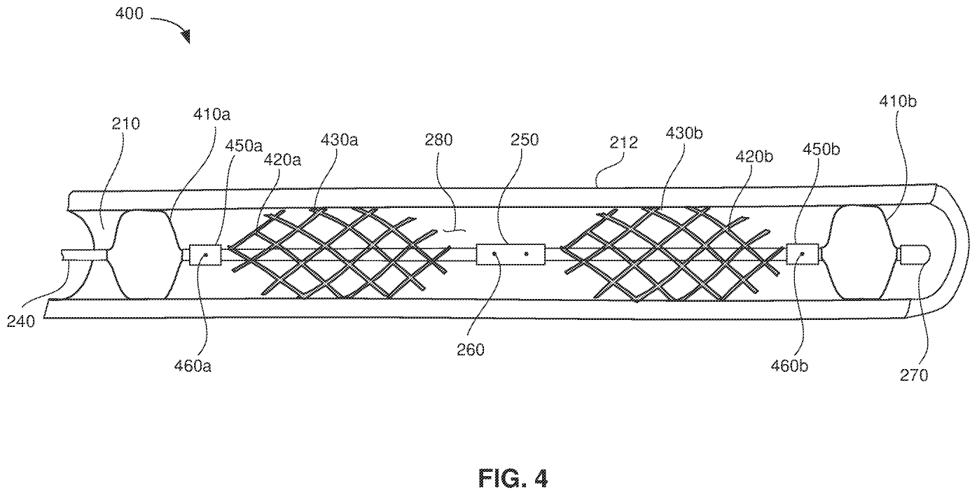

FIG. 4 shows another example of an applicator 400, in accordance with some implementations. Similar to the applicator 200 of FIG. 2, the applicator 400 may include the proximal electrode 430a, the distal electrode 430b, the elongate body 240, the sleeve 250, one or more outlets and/or inlets 260 and the atraumatic tip 270. In this example the proximal (first) electrode 430a is integral with the first expandable member 420a and the distal (e.g., second) electrode 430b is integral with the second expandable member 420b. for the first and second expandable members 420a and 420b may be formed of a mesh of wires, and the electrodes may be formed of one or more wires that also forms the expandable mesh. Additionally, the applicator 400 may optionally include a proximal occluder (expandable member 410a), a distal occluder (expandable member 410b), a proximal sleeve 450a, and a distal sleeve 450b.

The proximal and distal expandable members 410a and 410b (that may be also referred to as occluders) may be similar to the proximal and distal expandable members 220a and 220b of the applicator 200. For example, the proximal and distal expandable members 410a and 410b may be formed from any conformable and expandable material. Furthermore, the proximal and distal expandable members 410a and 410b may be inflated, enlarged, or expanded by any feasible liquid or gas. The proximal and distal expandable members 410a and 410b may be used to form a leak-resistant seal in and around the treatment area bounded by the proximal and distal expandable members 410a and 410b.

In some embodiments, the proximal and distal electrodes 430a and 430b may be coupled to the elongate body 240 and expanded by mechanical means, such as but not limited to push-pull tendons (not shown) that may be included or enclosed within the elongate body 240. For example, the push-pull tendons may be actuated by the elongate applicator tool 102 to cause the proximal and distal electrodes 430a and 430b to expand and circumferentially contact the walls of the lumen 210 and to collapse and/or contract away from the walls of the lumen 210. The proximal and distal electrodes 430a and 430b may otherwise be similar to the proximal and distal electrodes 230a and 230b of the applicator 200.

The proximal and distal sleeves 450a and 450b may be similar to the sleeve 250 of the applicator 200. The proximal and distal sleeves 450a and 450b may include one or more proximal and distal outlets and/or inlets 460a and 460b, respectively. Although a single outlet (and/or inlet) is shown, the proximal and distal sleeves 450a and 450b may include any feasible number of outlets and/or inlets. The proximal and distal outlets and/or inlets 460a and 460b, together or alternately with the outlets and/or inlets 260, may enable the delivery and removal of the filling material 280 to the treatment area.

Similar to as described with respect to the applicator 200, the applicator 400 may be positioned within the lumen 210 having a wall 212. The position of the applicator 400 may be verified, and the proximal and distal isolated expandable members 410a and 410b may be inflated and/or expanded to form a leak-resistant seal with the lumen 210. The proximal and distal electrodes 430a and 430b may then be expanded to circumferentially contact the inner wall of the lumen 210. The conductive liquid 280 may be infused to the treatment area through the outlets and/or inlets 260, the proximal outlets and/or inlets 460a, and/or the distal outlets and/or inlets 460b.

After infusion of the conductive liquid 280, a nanosecond pulsed electrical field treatment may be provided through the proximal and distal electrodes 430a and 430b. Following the nanosecond pulsed electrical field treatment, the filling material (e.g., conductive liquid) 280 may be removed through the outlets and/or inlets 260, the proximal outlets and/or inlets 460a, and/or the distal outlets and/or inlets 640b. The proximal and distal isolated expandable members 410a and 410b and the proximal and distal electrodes 430a and 430b may be collapsed or retracted, and the applicator 400 may be moved to another treatment area or withdrawn from the patient.

Also, in some variations (not shown), for example, using monopolar electrodes, the applicator 400 may include a single expandable member 420 comprising monopolar electrode(s) (instead of the proximal and distal isolated expandable members 420a and 420b). The expandable members/occluders 410a and 410b may surround distally and proximally the expandable member 420 with monopolar electrode(s). The expandable members 410a and 410b may be inflated and/or expanded to form a leak-resistant seal in and around the treatment area bounded by the proximal and distal expandable members 410a and 410b. The conductive liquid 280 may be infused to the treatment area through the outlets and/or inlets that may be located along the elongated body 240, for example, between the expandable member 410a and 410b, including between the expandable member 410a and the expandable member 420 or between the expandable member 410b and the expandable member 420.

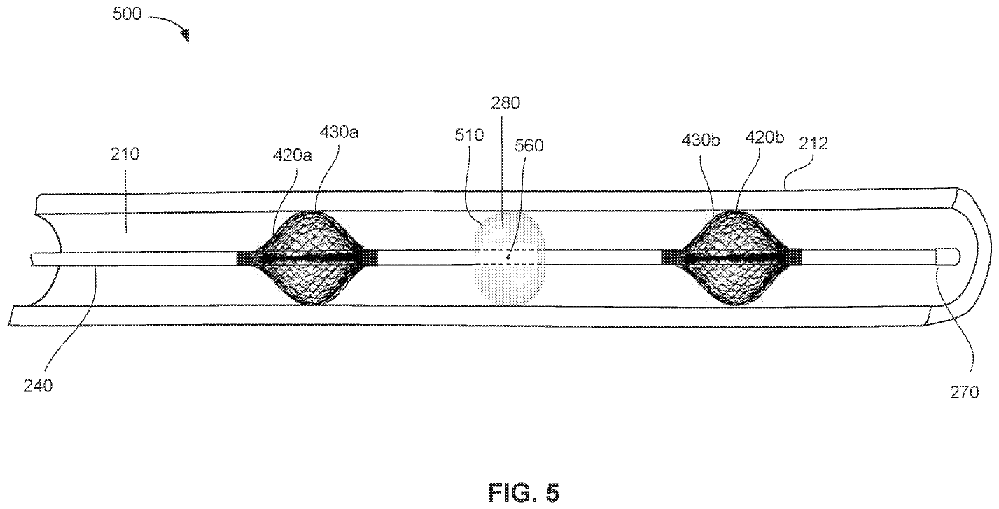

FIG. 5 shows another example of an applicator 500, in accordance with some embodiments. The applicator 500 may include the catheter 240, the atraumatic tip 270 (as shown in FIG. 2), and the proximal and distal electrodes 430a and 430b (as shown in FIG. 4), which may be formed on expandable members 420a, 420b or may be part of the expandable members. As described in FIG. 3, above, the electrodes may be formed as a part of a mesh (e.g., knitted, woven, braided, etc.) wire material that is at least partially un-insulated to form the active electrode region that contacts the wall of the lumen when expanded. The applicator 500 may include an additional expandable member 510 (e.g., a third expandable member) disposed between the electrodes 430a and 430b that forms the filling material to reduce or prevent arcing within the body lumen between the electrodes. The expandable member 510 may be a balloon that is filled, for example, with a fluid through one or more ports 560 within the expandable member. When inflated, the balloon 510 forms a seal with the wall of the body lumen and blocks an air path between the electrodes 430a and 430b.

The expandable member 510 may be similar to the proximal and distal occluders (expandable members) 410a and 410b. Thus, the expandable member 510 may be coupled to the elongated body, such as catheter 240, and configured to expand to contact the wall 212 of the lumen 210. However, if the balloon 510 is filled, for example, just with air or other non-conductive fluid, when high voltages are used the arcing may still occur under some circumstances and cause a destruction of the balloon 510. It was discovered, however, that filling or inflating the balloon 510 with a filling material as described herein may enhances ablation and reduce or eliminate arcing between the electrodes. For example, when the balloon 510 is inflated/filled, for example, with saline, no arcing and destruction of the balloon occurs. Therefore, in some variations, the expandable member 510 may be expanded by a conductive liquid (e.g., saline) delivered to the interior of the expandable member 510 through the outlets and/or inlets 560. In this example, the filling material is therefore the saline (or other filling material) contained within the expandable member (balloon).

In some embodiments, when the expandable member 510 is expanded to circumferentially contact and seal the inner wall of the lumen 210, the expandable member 510 may create a barrier and prevent or reduce electrical arcing between the proximal and distal electrodes 430a and 430b. In this manner, the expandable member 510 may enhance tissue ablation associated with the nanosecond pulsed electrical field treatment within the treatment area. In some variations, contact with the lumen 210 may be verified by measuring a pressure of the liquid or gas used to expand the expandable member 510.

In a manner similar to as described with respect to the applicator 200, the applicator 500 may be positioned within the lumen 210. The position of the applicator 500 may be verified, and the expandable member 510 may be expanded to contact the inner wall of the lumen 210. Next, the proximal and distal electrodes 430a and 430b may be placed into contact with the inner wall of the lumen 210 circumferentially by expanding the expandable members 420a, 420b. A nanosecond pulsed electrical field treatment may be provided through the proximal and distal electrodes 430a and 430b. Following the treatment, the proximal and distal electrodes 430a and 430b and the filling material (expandable member 510) may be collapsed or retracted. In some variations, the filling material (expandable member 510) may be collapsed or retracted by a removal of liquid through the outlets and/or inlets 560. The applicator 500 may be moved to another treatment area or withdrawn from the patient.

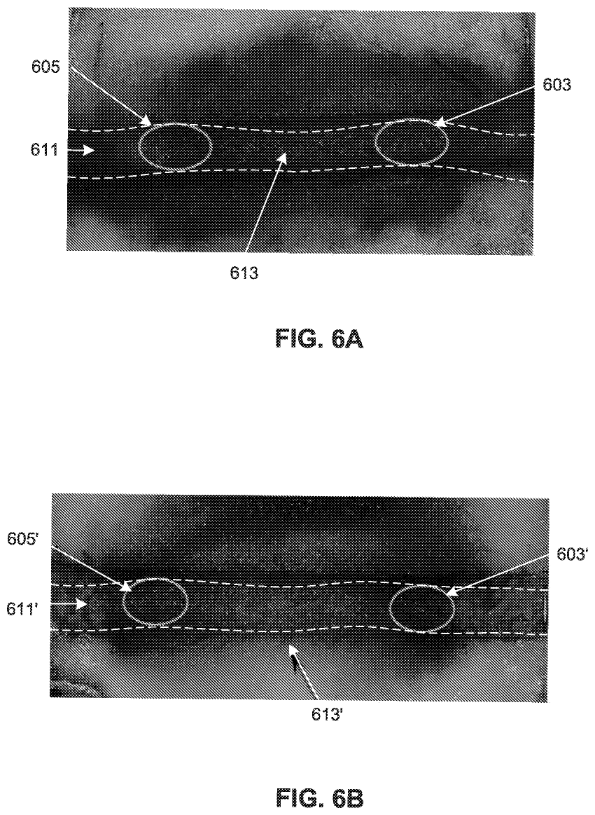

FIGS. 6A-6B illustrate examples of the operation of an applicator similar to that shown in FIG. 3 within a tissue model. In FIG. 6A the device was inserted into a lumen region 611 (shown between the dashed lines) within the tissue model. The locations of the first and second electrodes are shown by the white circles 603, 605. In FIG. 6A saline was added to the region 613 between the first and second electrodes before applying the pulsed electrical fields. The field was pulsed in the sub-microsecond range and resulted in a broadly effected region both around the electrodes (e.g., within the walls of the lumen) as well as the region between the electrodes. In contrast, in FIG. 6B a similar model including a lumen 611′ was used with the same applicator (similar to that shown in FIG. 3), but without adding saline to the region 613′ between the first electrode 605′ and the second electrode 603′. In FIG. 6B the area between the electrodes 613′ was much less effected by the electrical field resulting from the same sub-microsecond pulsing and had a much shallower penetration of the electrical field overall.

A prototype apparatus such as the one shown in FIG. 5, including an expandable member that can be expanded to circumferentially contact the inner wall of a lumen, was also tested in a tissue model such as the one shown in FIGS. 6A-6B. The test lumen was approximately 6 mm in diameter. When the device was used to apply sub-microsecond pulsing, with the expandable member (e.g., balloon) between the electrodes un-inflated, arcing was observed. After inflating the expandable member so that it could act as a filling material between the first and second electrodes (or sets of electrodes), arcing did not occur until the energy applied was nearly double that at which arcing occurred without the expandable member. The effect was reversible, and removing (e.g., by collapsing/deflating) the expandable member again resulted in arcing between the electrodes.

### Methods to Provide Pulse Energy Treatment

In general, the methods and apparatuses described herein may provide an electrical energy (e.g., microsecond, sub-microsecond, nanosecond, etc., pulsed electrical energy) treatment to various body cavities, lumens, pathways or any other feasible lumina.

In some embodiments, the pulsed electrical energy treatment may be provided in the presence of a filling material that is infused to an area of the body lumen to receive the treatment. The conductive liquid may enhance tissue ablation resulting from the pulsed electrical energy treatment, particular for regions of the body lumen that may lack a natural conductive liquid, such as blood, or the like.

In some other embodiments, the filling material may include an expandable member (such as a balloon or other container) that may be disposed between at least two electrodes that contact the lumen. Presence of the filling material may prevent or reduce electrical arcing between the at least two electrodes, thereby enhancing tissue ablation resulting from the pulsed electrical energy treatment.

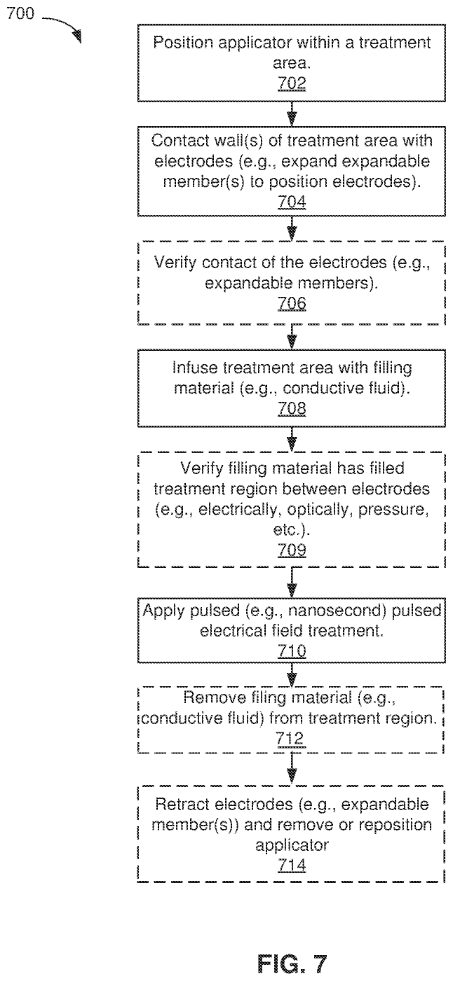

FIG. 7 is a flowchart depicting an example of one method 700 for applying a pulsed electrical treatment to a patient. Some examples may perform the operations described herein with additional operations, fewer operations, operations in a different order, operations in parallel, and some operations differently. The method 700 may be used with the applicator 200 of FIG. 2, the applicator 300 of FIG. 3, the applicator 400 of FIG. 4, or any other feasible applicator.

In FIG. 7, the method 700 may begin as an applicator is positioned within a treatment area (block 702). For example, the system 100 of FIG. 1 may be used to position an applicator (such as, but not limited to any of the applicators of FIGS. 2-4) within an identified treatment area. Any appropriate treatment area (e.g. body lumen) may be treated.

Next, expandable members of the applicator may be expanded (block 704) to place the electrodes (or sets of electrodes) in contact with the lumen walls. For example, if the applicator includes one or more expandable members, such as balloons, wire mesh, or the like, the one or more expandable members may be expanded to contact the electrodes to the patient's body lumen. In some variations, the one or more expandable members may expand to place the electrodes into contact with an inner wall of the body lumen. A fluid may be used to expand (inflate) the expandable members, or they may be mechanically expanded. Alternatively, the applicator may include one or more expandable electrodes that may be expanded to contact the body lumen without a separate expandable member. In some variations, the expandable members positioning the electrodes may be expanded with guide wires, tendons, or the like. The electrodes may be disposed, surround, or otherwise be coupled to expandable members and expand as the expandable member expands.

Contact between the electrodes and the body lumen may optionally be verified (block 706). This optional operation is depicted with dashed lines in FIG. 7. In variations in which the region between the electrodes is occluded, e.g., by the same expandable member that position the electrodes or by separate occluders (expandable members), the proper (e.g., leak-resistant) contact between the occluders and the lumen may be verified by measuring or monitoring the pressure of the fluid or gas used to expand the one or more expandable member. In some other variations, when the electrodes are disposed on the expandable members, contact of the electrodes may be verified by measuring an impedance using the electrodes. In still other variations, a camera or other optical device may be used to verify contact between the electrodes and the lumen. If proper contact is not present, then adjustments to the applicator position or inflation pressure may be made.

The treatment area may be infused with a filling material (block 708). For example, the filling material may be infused through inlet(s) (e.g., infusion ports) disposed on the elongate body into an area between (bounded by) two or more occluders. Optionally, no occluders may be used or a single occluder may be used. In some variations, the filling material may also be used to expand the one or more expandable members. In some other variations, the filling material may be saline, a contrast dye, a hypertonic solution, a hypotonic solution, a combination thereof, or any other feasible liquid.

The appropriate extent of the filling material may be optionally verified (block 709). In some variations, a pressure of the filling material that has been infused may be monitored or measured. If a proper (e.g., leak-resistant) contact between the one or more occluders and the body lumen exists, then the pressure of the filling material may remain relatively constant. If proper contact is not present, then adjustments to the applicator position or inflation pressure may be made.

The pulsed (e.g. nanosecond pulsed) electrical field treatment may be applied (block 710). For example, nanosecond pulse electrical field signals may be coupled to the electrodes that are in contact with the patient. Presence of the filling material may enhance tissue treatment by the pulsed electrical field treatment, and particularly sub-microsecond (e.g., nanosecond) pulsed treatment.

The filling material may be removed from the treatment area (block 712) following the application of pulsed energy. For example, the filling material may be pumped out from the treatment area through the outlets and/or inlets used to deliver the filling material.

The expandable members of the applicator may be retracted (block 714). For example, the fluid used to expand the expandable members may be pumped out of the expandable members to retract, compress, collapse, and/or contract them into or onto the elongate body. In another example, tendons or guide wires may be used to retract, compress, collapse, and/or contract an expandable member and/or electrodes.

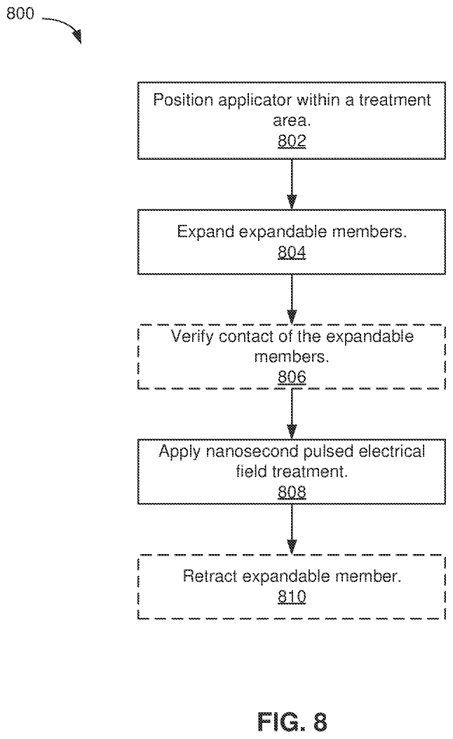

FIG. 8 is a flowchart depicting another example of a method 800 for applying a pulsed electrical treatment to a patient. Some examples may perform the operations described herein with additional operations, fewer operations, operations in a different order, operations in parallel, and some operations differently. The method 800 may be used with the applicator 500 of FIG. 5, the applicator 600 of FIG. 6, or any other feasible applicator.

In FIG. 8, the method 800 may begin as an applicator is positioned within a treatment area in the block 802. Operations of block 802 may be similar to those of block 702 described in FIG. 7.

The expandable members of the applicator are expanded in block 804. For example, if the applicator includes an expandable member (such as the expandable member 510) and/or the expandable members/electrodes 430a/420a and 430b/420b of FIG. 5, then the expandable members may be expanded to contact a body lumen. In some variations, the expandable member may be expanded by a fluid. In some other variations, the expandable member may be expanded by tendons, guide wires, or the like.

Contact of the one or more expandable members to the patient's body may be optionally verified in block 806. This optional operation is depicted with dashed lines in FIG. 8. For example, the contact between the one or more expandable members and the lumen may be determined by monitoring or measuring a pressure of the fluid used to expand the expandable member, or by measuring an impedance between at least two electrodes.

The nanosecond pulse electrical field treatment is applied in block 808. For example, nanosecond pulse electrical field signals may be coupled to the electrodes that are in contact with the lumen. In some embodiments, presence of an expandable member (such as expandable member 510) between at least two electrodes may prevent or reduce electrical arcing associated with the nanosecond pulse electrical field treatment.

The expandable members of the applicator may be retracted in block 810. For example, the fluid or gas used to expand the expandable members may be pumped out of the expandable members to retract, compress, collapse, and/or contract them into or onto the elongate body. In another example, tendons or guide wires may be used to retract, compress, and/or contract an expandable member or electrode.

As mentioned above, any of the apparatuses described herein may be implemented in robotic systems that may be used to position and/or control the electrodes during a treatment. For example, a robotic system may include a movable (robotic) arm to which elongate applicator tool is coupled. Various motors and other movement devices may be incorporated to enable fine movements of an operating tip of the elongate applicator tool in multiple directions. The robotic system and/or elongate applicator tool may further include at least one image acquisition device (and preferably two for stereo vision, or more) which may be mounted in a fixed position or coupled (directly or indirectly) to a robotic arm or other controllable motion device. In some examples, the image acquisition device(s) may be incorporated into the elongate applicator tool.

Examples of the methods of the present disclosure may be implemented using computer software, firmware, or hardware. Various programming languages and operating systems may be used to implement the present disclosure. The program that runs the method and system may include a separate program code including a set of instructions for performing a desired operation or may include a plurality of modules that perform such sub-operations of an operation or may be part of a single module of a larger program providing the operation. The modular construction facilitates adding, deleting, updating and/or amending the modules therein and/or features within the modules.

In some examples, a user may select a particular method or example of this application, and the processor will run a program or algorithm associated with the selected method. In certain examples, various types of position sensors may be used. For example, in certain example, a non-optical encoder may be used where a voltage level or polarity may be adjusted as a function of encoder signal feedback to achieve a desired angle, speed, or force.

Certain examples may relate to a machine-readable medium (e.g., computer-readable media) or computer program products that include program instructions and/or data (including data structures) for performing various computer-implemented operations. A machine-readable medium may be used to store software and data which causes the system to perform methods of the present disclosure. The above-mentioned machine-readable medium may include any suitable medium capable of storing and transmitting information in a form accessible by processing device, for example, a computer. Some examples of the machine-readable medium include, but not limited to, magnetic disc storage such as hard disks, floppy disks, magnetic tapes. It may also include a flash memory device, optical storage, random access memory, etc. The data and program instructions may also be embodied on a carrier wave or other transport medium. Examples of program instructions include both machine code, such as produced by a compiler, and files containing higher level code that may be executed using an interpreter.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to perform or control performing of any of the steps, including but not limited to: displaying, communicating with the user, analyzing, modifying parameters (including timing, frequency, intensity, etc.), determining, alerting, or the like. In some exemplary examples, hardware may be used in combination with software instructions to implement the present disclosure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “mounted”, “connected”, “attached” or “coupled” to another feature or element, it can be directly mounted, connected, attached, or coupled to the other feature or element or intervening features or elements may be present. In contrast, when a feature or element is referred to as being “directly mounted”, “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting. For example, as used herein, the singular forms “a”, “an” and “the” are intended to include the plural forms as well, unless the context clearly indicates otherwise. As used herein, the term “and/or” includes any and all combinations of one or more of the associated listed items.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present apparatuses and methods.

The terms “comprises” and/or “comprising,” when used in this specification (including the claims), specify the presence of stated features, steps, operations, elements, and/or components, but do not preclude the presence or addition of one or more other features, steps, operations, elements, components, and/or groups thereof. Unless the context requires otherwise, “comprise”, and variations such as “comprises” and “comprising,” means various components can be co-jointly employed in the methods and articles (e.g., compositions and apparatuses including device and methods). For example, the term “comprising” will be understood to imply the inclusion of any stated elements or steps but not the exclusion of any other elements or steps.

Any of the apparatuses and methods described herein may include all or a sub-set of the components and/or steps, and these components or steps may be either non-exclusive (e.g., may include additional components and/or steps) or in some variations may be exclusive, and therefore may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. Although various illustrative examples are described above, any of a number of changes may be made to various examples without departing from the scope of the disclosure as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the apparatuses and methods as it is set forth in the claims.

Various examples may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

1.-42. (canceled)

43. An apparatus for delivering electrical fields, the apparatus comprising:
an elongate body;
a first expandable member at a distal end region of the elongate body;
a first electrode on or forming at least a part of the first expandable member, the first electrode is configured to apply high field strength electric field;
a second expandable member at the distal end region of the elongate body and laterally offset from the first expandable member;
a second electrode on or forming at least a part of the second expandable member, the second electrode is configured to apply high field strength electric field;
an outlet configured to deliver a filling material between the first electrode and the second electrode, wherein the filling material is configured to prevent or disrupt arcing between the first and the second electrodes.

44. The apparatus of claim 43, further comprising a pulse generator configured to deliver a nanosecond pulsed electrical energy to the first electrode and the second electrode.

45. The apparatus of claim 43, further comprising one or more occluder configured to occlude a body lumen when deployed in the body lumen to prevent loss or leakage of the filling material from an area between the first and the second electrodes.

46. The apparatus of claim 45, wherein the one or more occluder comprises a first occluder and a second occluder laterally offset from the first occluder so that the first and the second electrodes are between the first and the second occluders and the first and the second occluders form an enclosed space within the body lumen when deployed in the body lumen.

47. The apparatus of claim 46, wherein the first occluder is formed on the first expandable member.

48. The apparatus of claim 43, wherein the filling material comprises at least one of: a saline, a contrast dye, a hypertonic solution, a hypotonic solution, a solid material, a general conductive liquid, or a combination thereof.

49. The apparatus of claim 45, wherein the one or more occluder is positioned between the first and the second electrodes.

50. The apparatus of claim 45, wherein the one or more occluder comprises a balloon configured to be inflated with the filling material from the outlet.

51. The apparatus of claim 43, wherein the outlet comprises one or more outlets and wherein at least one of the one or more outlets is positioned on the first expandable member, on the second expandable member, or along a length of the elongated body between the first expandable member and the second expandable member.

52. An apparatus for delivering electrical fields, the apparatus comprising:
an elongate body;
a first expandable member at a distal end region of the elongate body;
a first electrode on or forming at least a part of the first expandable member;
a second expandable member at the distal end region of the elongate body and laterally offset from the first expandable member;
a second electrode on or forming at least a part of the second expandable member; and
an outlet configured to deliver a filling material between the first electrode and the second electrode.

53. The apparatus of claim 52, wherein one or both of the first expandable member and the second expandable member comprises a balloon, a mesh, or expandable struts.

54. The apparatus of claim 52, further comprising a reservoir of filling material in communication with the outlet.

55. The apparatus of claim 52, wherein the filling material comprises one or more of: a saline, a contrast dye, a hypertonic solution, a hypotonic solution, a solid material, a general conductive liquid, or a combination thereof.

56. The apparatus of claim 52, further comprising one or more controller and a pulse generator configured to deliver electrical energy to the first and second electrodes, wherein the one or more controller is configured to perform at least one of the following: deploying the filling material out of the outlet, controlling delivery of the electrical fields between the first and second electrodes, and/or coordinating an application of the electrical fields and deployment of the filling material.

57. The apparatus of claim 56, wherein at least one of the one or more controller is further configured to confirm that the filling material has filled a space within a body lumen between the first electrode and the second electrode or that a target amount of the filling material was introduced into the body lumen.

58. The apparatus of claim 57, wherein the at least one of the one or more controller is configured to confirm that the filling material has filled the space by detecting one or more of: a pressure, an impedance, or a contrast dye.

59. The apparatus of claim 57, wherein the at least one or a different controller of the one or more controller is configured to coordinate an application of pulsed electrical energy so that the pulsed electrical energy is applied after the filling material has been deployed from the outlet.

60. The apparatus of claim 56, wherein the pulse generator is configured to apply a nanosecond pulsed electrical energy.

61. The apparatus of claim 52, further comprising a third expandable member positioned between the first expandable member and the second expandable member such that the outlet is positioned to deliver the filling material inside the third expandable member.

62. The apparatus of claim 61, the apparatus comprises one or more controller configured to confirm that: 1) the filling material has filled a space within the third expandable member sufficient to expand the third expandable member to form a seal with a wall of a body lumen when the apparatus is deployed within the body lumen.

63. An apparatus for delivering electrical fields, the apparatus comprising:
an elongate body;
a first expandable member at a distal end region of the elongate body;
a first electrode on or forming at least a part of the first expandable member;
a second expandable member at the distal end region of the elongate body and laterally offset from the first expandable member;
a second electrode on or forming at least a part of the second expandable member;
an outlet configured to deliver a filling material between the first electrode and the second electrode; and
a third expandable member positioned between the first expandable member and the second expandable member such that the outlet is positioned to deliver the filling material inside the third expandable member.

64. The apparatus of claim 63, wherein the third expandable member comprises a balloon.
